(19) United States
(12) Patent Application Publication
(10) Pub. No.: US 2014/0107692 A1
(43) Pub. Date: Apr. 17, 2014

(54) METHOD OF TREATING A LUMEN REGION OF A SUBJECT

(71) Applicant: OLYMPUS MEDICAL SYSTEMS CORP., Tokyo (JP)

(72) Inventors: Nobuko MATSUO, Tokyo (JP); Takahiro KOGASAKA, Hachioji-shi (JP); Kazuo BANJU, Hachioji-shi (JP); Koji NISHIZAWA, Hachioji-shi (JP); Takayasu MIKKAICHI, Fuchu-shi (JP)

(73) Assignee: OLYMPUS MEDICAL SYSTEMS CORP., Tokyo (JP)

(21) Appl. No.: 13/653,998

(22) Filed: Oct. 17, 2012

(51) Int. Cl. A61M 29/02

(52) U.S. Cl. 606/192

(57) ABSTRACT

A method of treating a lesion of a tubular organ that communicates with a natural orifice of a subject is provided. In the method, a channel of the tubular organ is closed or substantially closed at a desired first position on a far side of the lesion. Then, the channel is closed or substantially closed at a second position near the orifice, i.e. on a near side of the lesion. Then, the tubular organ is linearized by charging a fluid into a closed space formed between the first and second positions by bringing the tubular organ into a closed or substantially closed state. Treatment tools are inserted into the linearized closed space to treat the lesion.

### BACKGROUND

1. Technical Field

The present invention relates to a method of treating a lumen (i.e., tubular cavity) region, such as the rectum, of a subject, and in particular to a method of treating a lesion located in an inner wall of a lumen region of a subject, by inserting a rigid tubular member into the lumen region, and then inserting treatment tools into the lumen region via the tubular member under observation of endoscopic images.

2. Related Art

Various surgical procedures are available for removing a tumor or the like located in a region of the rectum/colon of a subject. As one of such surgical procedures, TEM (Transanal Endoscopic Microsurgery) is well known. TEM makes use of a rigid and linear sheath having a length of some degree (about 75 to 200 mm). The sheath is inserted into the anus of a subject, while being fixed to the bed via a holder. A rigid scope and treatment tools, such as forceps, are inserted into the sheath to provide treatment using the treatment tools under observation of images picked up by the rigid scope.

The sheath used in TEM has a length sufficient for the sheath to reach a point immediately before a lesion, such as a cancerous tumor. Thus, the lesion is treated in a state where the lesion is captured forward of the sheath. Accordingly, the surgical field (treatment range) in TEM is restricted by the dimension of the head portion of the sheath in the radial and depth (longitudinal) directions. Specifically, regarding the radial direction, the surgical field is restricted to only 180 degrees on the bottom surface of the rectum/colon due to the interference between the sheath and the forceps. Regarding the depth direction, the surgical field is restricted to a straight range that can be provided by the straight sheath, i.e. a straight range up to an S-shaped portion of the rectum. A treatment range is determined by these restrictions.

### SUMMARY

Under the conditions as set forth above, there has been a desperate need of expanding the treatment range, i.e. expanding the treatment range determined by the dimension of the sheath in the circumferential and depth directions (i.e. the treatment range of the tubular organ in the circumferential and longitudinal directions).

According to a typical embodiment, a method of treating a lesion of a tubular organ that communicates with a natural orifice of a subject is provided. The method includes: a first step of closing or substantially closing a channel of the tubular organ at a desired first position on a far side of the lesion; a second step of closing or substantially closing a channel of the tubular organ at a second position on a near side of the lesion, the second position being near the natural orifice; a third step of linearizing the tubular organ by sending a fluid into a closed space of the tubular organ, the closed space being created between the first and second positions by bringing the tubular organ into a closed or substantially closed state through the first and second steps; and a fourth step of treating the lesion by inserting treatment tools into the linearized closed space.

Thus, in the case where the channel of the tubular organ is curved like the sigmoid colon, for example, the degree of the curve is reduced by creating and linearizing the closed space. In other words, the creation and linearization of the closed space can ensure a wider surgical field having good visibility.

### BRIEF DESCRIPTION OF THE DRAWINGS

In the accompanying drawings:

FIG. 1 is a partially broken cross-sectional view illustrating a treatment system in general for carrying out a method of treatment, according to a first embodiment of the present invention;

FIG. 2 is a cross-sectional view illustrating an example of an obturator used in the treatment system;

FIGS. 3A and 3B are side and front views, respectively, illustrating a faceplate used in the treatment system;

FIGS. 4 to 10, FIG. 11A and 11B, and FIGS. 12A and 12B are diagrams illustrating, as examples, procedures and advantageous effects of the method of treatment according to the first embodiment and a method of treatment, according to a second embodiment of the present invention;

FIG. 13, FIGS. 14A and 14B, and FIG. 15 are diagrams illustrating modifications of the first embodiment;

FIG. 16 is a partially broken cross-sectional view illustrating a part of a treatment system for carrying out a method of treatment according to the second embodiment of the present invention;

FIGS. 17 and 18 are diagrams illustrating a process of charging/discharging gas to/from a second balloon, according to the second embodiment;

FIG. 19 is a diagram illustrating a modification of the second embodiment; and

FIG. 20 is a diagram illustrating another modification of the second embodiment.

### DETAILED DESCRIPTION OF THE PREFERRED EMBODIMENTS

With reference to the accompanying drawings, hereinafter are described some preferred embodiments of a method of treating a lumen region of a subject and a treatment system for carrying out the method, according to the present invention.

In the present embodiment, a typical region to be treated is the large intestine that communicates with the anus as a natural orifice of a subject.

In the embodiments set forth below, a method of treatment and a treatment system for carrying out the method are described. The method is used for treating a lesion that has occurred in the sigmoid colon or in a region located deeper than the sigmoid colon, such as the descending colon, which is a part of the large intestine as a tubular organ of a subject.

### First Embodiment

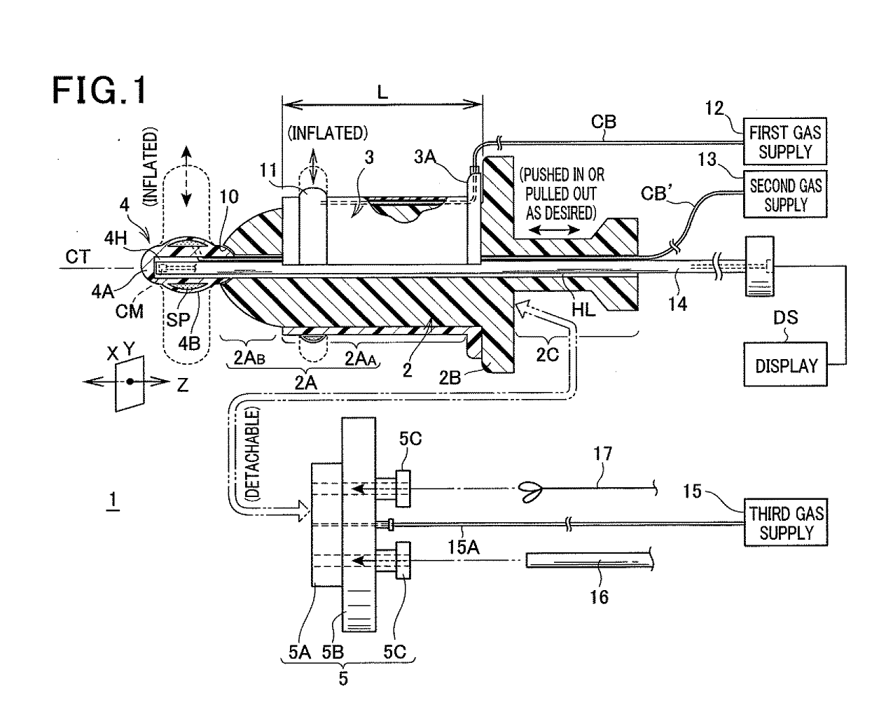

Referring to FIGS. 1 to 10, FIGS. 11A and 11B, and FIGS. 12A and 12B, hereinafter is described a first embodiment of the present invention.

First, a treatment system of the first embodiment is described. FIG. 1 is a partially broken cross-sectional view illustrating the treatment system in general for cutting away a method of treatment, according to the first embodiment. As shown in FIG. 1, the treatment system includes an applicator 1 and treatment tools 17. The applicator 1 is used for sending gas, such as CO₂, in advance, to a tubular space of the large intestine that includes a lesion (e.g., tumor) to apply pressure to the space. The treatment tools 17 (specifically described later) are used for treating the lesion located in the space, after sending the gas to the space and pressurizing the space using the applicator 1.

As shown in FIG. 1, the applicator 1 includes an obturator 2, a sheath 3, a second balloon 4 and a faceplate 5. The obturator 2 is substantially bullet-shaped. The sheath 3 is cylindrically shaped and has an inner space, into which a part of the obturator 2 is inserted. The second balloon 4 (specifically described later) as a closing member is detachably engaged with the head portion of the obturator 2. The faceplate 5 is partially insertable into the sheath 3.

Here, the longitudinal direction of the obturator 2, i.e. the longitudinal direction of the applicator 1, is defined to be an axial direction Z. A plane perpendicular to the axial direction Z is defined to be a perpendicular plane XY. An axis that passes through the center position of the perpendicular plane XY of the obturator 2 and extends in the axial direction Z is defined to be a center axis CT.

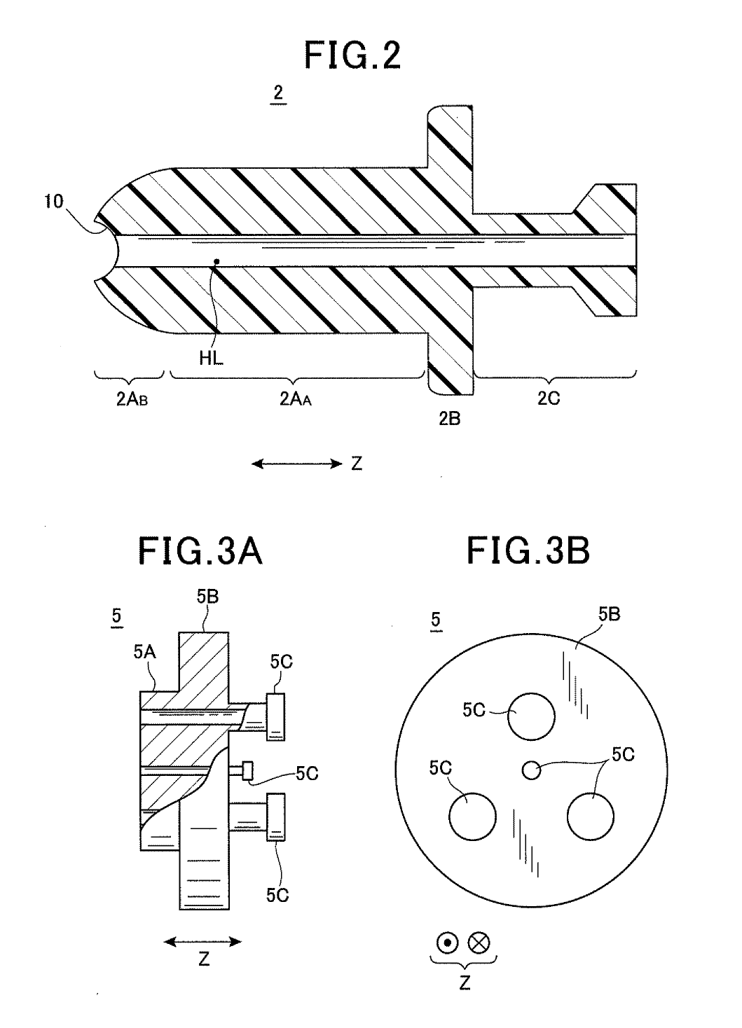

FIG. 2 is a cross-sectional view illustrating an example of the obturator 2. As shown in FIG. 2, the obturator 2 has a body 2A, a flange 2B and a guide 2C. The body 2A has substantially a cylindrical shape and has a predetermined length in the axial direction Z. The flange 2B is integrally provided at one end of the body 2A in the axial direction Z. The guide 2C is integrally projected from one side face of the flange 2B in the axial direction Z. The obturator 2 is monolithically formed as a whole using a resin material or the like.

The body 2A of the obturator 2 includes a base portion 2A_{A} and a head portion 2A_{B}. The base portion 2A_{A} has a cylindrical shape and extends in the axial direction Z from the flange 2B by a distance L. The head portion 2A_{B} is rounded and positioned at the extended end of the base portion 2A_{A}. Thus, the obturator 2 as a whole has substantially a bombshell-like shape. The head portion 2A_{B} is provided with a holding hole 10 that can hold the second balloon 4 as a closing member. The center position of the perpendicular plane XY of the holding hole 10 coincides with the center axis CT.

When the applicator 1 is inserted into the anus of a patient, the flange 2B is brought into contact with the external surface of the anus to function as a stopper. Thus, the flange 2B is formed being integrated into the body 2A and permitted to have a larger diameter.

The guide 2C is integrally formed with the flange 2B, being projected in the axial direction Z from the outer end face of the flange 2B. The guide 2C holds an endoscope, for example, which will be described later.

As shown in FIG. 2, the obturator 2 is provided, throughout its length along the axial direction Z, with a through hole HL. The through hole HL has a predetermined radius centering on the center axis CL. Accordingly, the through hole HL passes through the obturator 2, from the head end of the body 2A, i.e. the center of the holding hole 10, to the tail end of the guide 2C.

Further, the sheath 3 is formed into a cylindrical shape using, for example, a transparent or semitransparent resin material. The sheath 3 has a predetermined inner diameter and outer diameter (e.g., outer diameter of 40 mm) and an axial length L (e.g., 40 to 200 mm). The inner diameter of the sheath 3 is determined so that the body 2A of the obturator 2 can be intimately in contact with the sheath 3 but that the body 2A can be easily pushed into or pulled out of the sheath 3 as desired. The length L of the sheath 3 is determined so that the sheath 3 can cover the principal cylindrical portion of the body 2A of the obturator 2.

The sheath 3 has an outer peripheral surface having an end in the axial direction Z, which end is provided with a first balloon 11 that can be inflated/deflated with the charge/discharge of gas. For example, the position of the first balloon 11 is determined so that the first balloon 11 is located within about 40 mm from the head end of the sheath 3. In this case, the position is determined in relation to the length L of the sheath 3. The first balloon 11 is connected to a first gas supply 12 that can charge/discharge gas to/from the first balloon 11 via a gas charge/discharge tube CB. When the applicator 1 is inserted into the anus of a patient, the balloon is deflated, and after completing the insertion, inflated.

The outer peripheral surface of the sheath 3 has the other end in the axial direction Z, which has a larger diameter to form a stopper 3A. Thus, when inflated, the first balloon 11 cooperates with the stopper 3A and the flange 2B to sandwich the entrance portion of the anus from inside and outside. As a result, the sheath 3, i.e. the applicator 1, is held by the anus.

The second balloon 4 as a closing member is formed of a translucent elastic material that enables inflation/deflation of the balloon as desired with the charge/discharge of gas. The second balloon 4 has a balloon base portion 4A and an expansion/contraction portion 4B. The balloon base portion 4A has substantially an elliptical shape. The expansion/contraction portion 4B is integrally formed with the balloon base portion 4A so as to be located radially outside the base portion 4A. The expansion/contraction portion 4B has a hermetically hollow space SP and thus can be inflated/deflated in the radial direction. The hollow space SP is located radially outside the base portion 4A and isolated from the base portion 4A. The hollow space SP of the expansion/contraction portion 4B communicates with a second gas supply 13 via a gas charge/discharge tube CB′ passed through the through hole HL. Gas is charged to or discharged from the hollow space SP with the gas charge/discharge operation performed by the second gas supply 13. Thus, the expansion/contraction portion 4B is able to selectively inflate (as shown by the broken line in FIG. 1) or deflate (as shown by the solid line in FIG. 1).

The balloon base portion 4A has an end in the axial direction Z, in which an engagement hole 4H having a circular cross section is open. The engagement hole 4H is used for establishing a detachable engagement between a head portion of a rod-like flexible scope 14 as a conveyer device and the second balloon 4. The scope 14 includes a rigid scope, a flexible scope or a rigid scope having a bendable head. The scope 14 is connected to a display DS so that images captured by a camera CM provided at the head of the scope 14 can be displayed on the display DS. Accordingly, the operator is able to operate the forward and backward movement of the scope 14, observing the images captured by the scope 14.

Thus, the second balloon 4, when deflated, keeps a long oval form as a whole, with one end thereof being engaged with the obturator 2 via the holding hole 10. When the second balloon 4 is inflated, the center portion and the vicinity thereof of the expansion/contraction portion 4B in the longitudinal direction are expanded in the radial direction (see the broken line of FIG. 1). As a result, the second balloon 4 increases its diameter as shown in FIG. 1, taking a shape of a doughnut. When inflated, the second balloon 4 is ensured to have a diameter that allows the balloon 4 to be intimately in contact with the inner wall of the rectum.

In the following description, a position between an object (e.g., a lesion or a balloon) and the anus of a patient is referred to as a position “before” the object, and a position further into the rectum/colon than the object is referred to as a position “after” the object.

After insertion of the obturator 2 into the anus, the second balloon 4 is ensured to be separated from the obturator 2 with the operator's manipulation of the scope 14, for advancement deeper into the rectum. With the operator's manipulation of the scope 14, the second balloon 4 is conveyed forward and brought to a desired position in the rectum. During the conveyance, the expansion/contraction portion 4B of the second balloon 4 is contracted. When the second balloon 4 has reached the desired position, the expansion/contraction portion 4B of the second balloon 4 is expanded with the operator's manipulation. Thus, at the desired position, the second balloon 4 is brought into intimate contact with the inner wall of the rectum via the radially outer side face of the second balloon 4. As a result, the rectum is divided into a space after the second balloon 4 and a substantially hermetic space before the second balloon 4. In this case, the desired position is located past (located after or deeper than) the lesion, such as cancer, in the rectum.

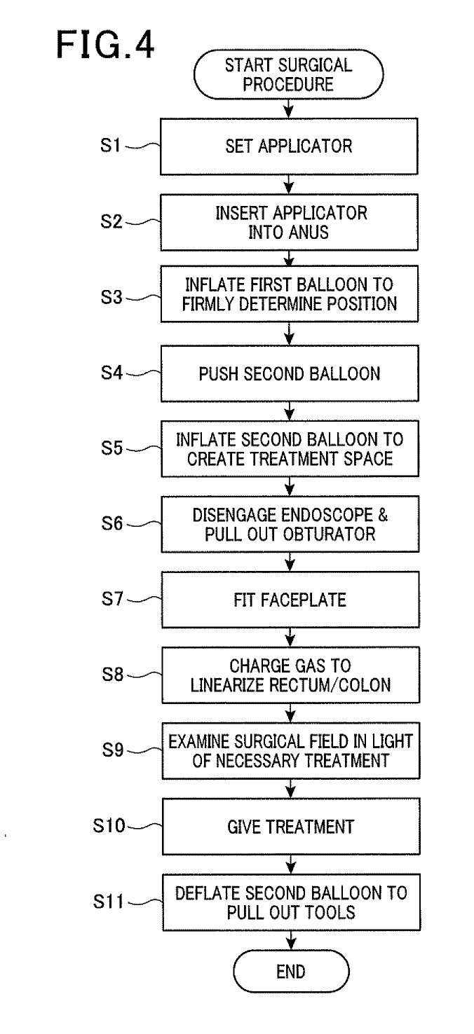

After pulling out the obturator 2 from the sheath 3, the faceplate 5 is hermetically mounted on the outer end portion of the sheath 3 in the axial direction Z. FIGS. 3A and 3B are side and front views, respectively, illustrating the faceplate 5. As shown in FIGS. 3A and 3B, the faceplate 5 has an insertion portion 5A, a flange portion 5B and a plurality of ports (lumens) 5C. The insertion portion 5A has a cylindrical shape and is hermetically inserted throughout its length, in the axial direction Z, into the sheath 3. The flange portion 5B is integrally formed with the insertion portion 5A and has a larger diameter than the insertion portion 5A. The plurality of ports 5C are integrally projected from the flange portion 5B. The faceplate 5 is made of a rigid or soft resin material. The plurality of ports 5C include a gas charge/discharge port which is connected to a third gas supply 15, and one or more treatment-tool ports into which a surgical scope 16 and treatment tools 17, such as forceps, are hermetically inserted. The ports 5C are provided with respective valves to ensure airtightness.

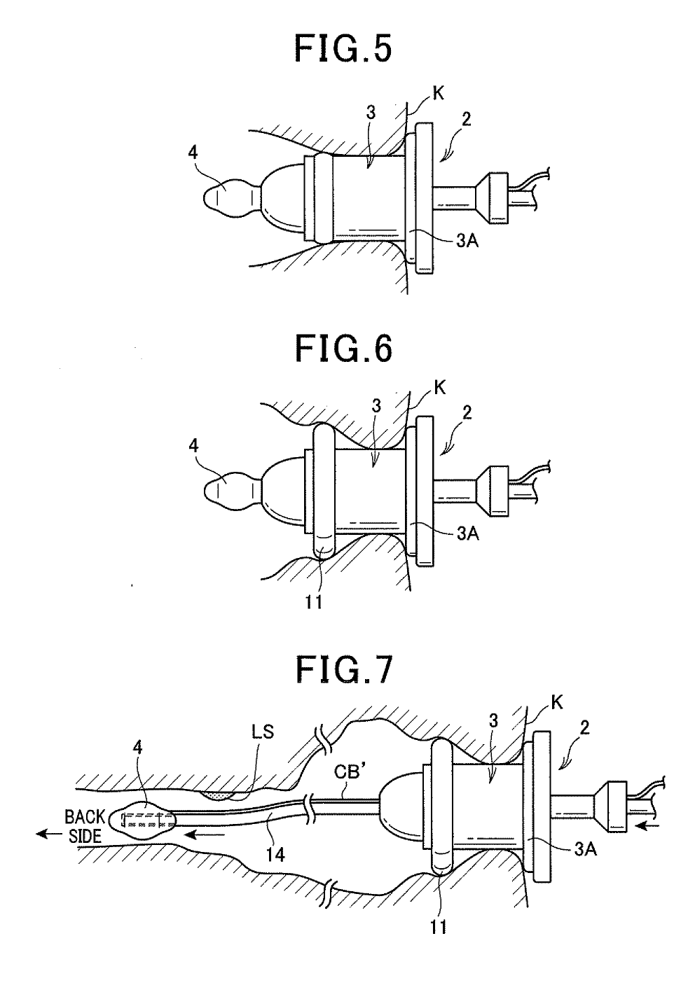

Referring to FIGS. 4 to 10, a treatment method using the treatment system is described. FIG. 4 is a flow diagram illustrating the procedure of treatment. FIGS. 5 to 10 are diagrams illustrating the treatment.

(1) First, the applicator 1 is set as shown in FIG. 1 (refer to step S1 of FIG. 4). Specifically, the obturator 2 is inserted into the sheath 3. Then, tubular members, i.e. the scope 14 and the gas charge/discharge tube CB′, are inserted into the through hole HL. In this state, the balloon 4 is set to the holding hole 10 at the head of the obturator 2. As a result, the applicator 1 is assembled as shown in FIG. 1.

(2) Then, the operator inserts the applicator 1 into an anus K of a patient so that the stopper 3A of the sheath 3 is brought into contact with the external surface of the anus K (refer to step S2 of FIG. 4, and FIG. 5).

(3) Then, the operator activates the first gas supply 12 to inflate the first balloon 11 on the sheath 3 (refer to step S3 of FIG. 4). Thus, the first balloon 11 comes into contact with the internal surface of the anus K (see FIG. 6). As a result, the stopper 3A and the first balloon 11 sandwich the anus K from inside and outside thereof to fix, or set, the sheath 3, or the whole applicator 1, to the anus K.

(4) After setting the applicator 1 to the anus K, the operator slowly pushes the scope 14 into the rectum together with the gas charge/discharge tube CB′, while observing the images captured by the scope 14 (refer to step S4 of FIG. 4, and FIG. 7). Thus, the second balloon 4 positioned at the head of the obturator 2 is gradually separated from the obturator 2 and moved forward. The operator pushes the tubular members so that the head of the scope 14, i.e. the second balloon 4, is brought to a desired position after a lesion LS. In the case where no images to be observed are available, the position of the lesion LS should be confirmed in advance using an endoscope or the like. Thus, in this case as well, the operator slowly pushes the tubular members to advance the second balloon 4 by a desired distance to a position after the lesion LS.

(5) After completing the pushing and positioning operation, the operator activates the second gas supply 13 to inflate the second balloon 4 as a closing member to a given extent (refer to step S5 of FIG. 4 and FIG. 8). Thus, as shown in FIG. 8, the second balloon 4 at the head is brought into intimate contact with the inner wall of the rectum and pushed radially outward to tightly engage with the rectum. As a result, being located at the desired position, the second balloon 4 separates the inner space of the rectum into a space after the balloon 4 and a substantially hermetically closed treatment space S before the balloon 4. Thus, with the sheath 3 and the obturator 2 being left at the entrance of the anus K, the substantially hermetically closed treatment space S is created between the second balloon 4 and the obturator 2 (sheath 3).

(6) When the treatment space S has been created, the operator disengages the scope 14 from the second balloon 4 and pulls the obturator 2 out of the sheath 3 (refer to step S6 of FIG. 4 and FIG. 9). Thus, the second balloon 4 remains in the rectum via the gas charge/discharge tube CB′.

(7) Then, the operator fits the faceplate 5 into the outer end portion of the sheath 3 (refer to step S7 of FIG. 4, and FIG. 10). In fitting the faceplate 5, the gas charge/discharge tube CB′ is ensured to hermetically pass through the gas charge/discharge port of the faceplate 5.

(8) Then, the operator connects a gas charge/discharge tube 15A of the third gas supply 15 to one of the ports 5C of the faceplate 5, i.e. to the port 5C as the gas charge/discharge port. At the same time, the operator activates the third gas supply 15 to charge gas, such as CO₂, into the treatment space S (refer to step S8 of FIG. 4, and Fit. 10). As a result, the treatment space S expands and is linearized by the pressure of the charged gas. In other words, if the treatment space S has a curved shape, as the sigmoid colon does, the degree of the curve of the treatment space S is reduced. Thus, the curved treatment space S is temporarily permitted to take an approximately linear shape.

Specifically, in the case where the treatment space S before sending the gas is substantially linear in the longitudinal direction, the treatment space S simply expands in the direction along the perpendicular plane XY of the applicator 1 with the pressure of the charged gas. As a result, the treatment space S expands as it is, substantially keeping a cylindrical shape. Thus, the space, i.e. surgical field, which can be used for treatment practice (which will be specifically described later) is enlarged to thereby further facilitate the treatment. For example, such a case corresponds to the case where the lesion LS is located comparatively near the anus K and the balloon 4 is positioned before the sigmoid colon.

On the other hand, in the case where the second balloon 4 is advanced past the sigmoid colon and positioned after the sigmoid colon, the treatment space S includes the sigmoid colon. Since the inner wall of the rectum is flexible, when such a curved portion is included in the treatment space S, the inner wall of the rectum is linearized receiving the radially outward pressure of the charged gas. The process of this linearization is schematically shown in FIGS. 11A and 11B. FIG. 11A illustrates that the second balloon 4 has been positioned after the tumor (the lesion LS) because the tumor is located in the vicinity of the sigmoid colon. In this case, the treatment space S includes the sigmoid colon, and accordingly, the treatment space S, as it is, substantially takes a shape of an S. However, the treatment space S in the present embodiment is created so as to be completely or substantially closed including the range covering the sigmoid colon and the tumor. Further, the treatment space S in the present embodiment is linearized with the application of a pressure caused by the gas supplied to the treatment space S.

It should be appreciated that the term “closed” here refers to a “state where a hermetic space is formed”. Further, the term “linearized” refers to not only a state of becoming completely straight but also a state obtained as a result of deforming a tubular organ from its original curved state to a more straightened state. The degree of linearization depends on the circumferential position of the lesion LS in the rectum, the distance from the anus K and the size of the surgical field required for the treatment.

(9) After linearizing the rectum, the operator inserts the surgical scope 16 into the treatment space S via one of the ports 5C of the faceplate 5. Using the surgical scope 16, the operator examines whether the size of the surgical field in the treatment space S is sufficient for the treatment practice, in light of the size and position of the lesion LS, the size of a spatial area necessary for the treatment, and the like (refer to step S9 of FIG. 4).

As a result of the examination, if the position of the second balloon 4, i.e. the position of the forefront end of the treatment space S, is too close to the lesion LS, the surgical scope 16 is pulled out and the scope 14 as a conveyer device is again inserted to adjust the position of the second balloon 4.

A scope serving both as the surgical scope 16 and the conveyer scope 14 may be used.

In inserting the surgical scope 16, gas may leak out of the treatment space S to reduce the internal pressure and accordingly reduce the linearity of the treatment space S. In such a case, the operator may charge gas from the third gas supply 15 to adjust the pressure inside the treatment space S. Alternatively, a pressure sensor (not shown) may be provided at the head end of the sheath 3 to automatically adjust pressure according to the information obtained from the pressure sensor.

(10) When it is determined that the surgical field is sufficiently ensured, the operator inserts the treatment tool 17, such as rigid forceps, via another one of the ports 5C to give necessary treatment to the lesion LS (refer to step S10 of FIG. 4). The reduction of linearity in the treatment space S in inserting the treatment tool 17 is remedied by charging gas in a manner similar to the above.

(11) After completing treatment, the first gas supply 12 is activated to deflate the second balloon 4. At the same time, the sheath 3 and the faceplate 5 are pulled out of the anus K together with the second balloon 4 (refer to step S11 of FIG. 4).

In this way, according to the present embodiment, the operator is able to obtain the linearized treatment space S as schematically shown in FIG. 12A. At the same time, the operator is able obtain a wide surgical field with its diameter being increased substantially along the perpendicular plane XY. Thus, a wide range is ensured along the radial direction of the rectum for the movement of the treatment tools, such as forceps, thereby facilitating treatment.

FIG. 12B comparatively illustrates an inconvenience of the conventional TEM (Transanal Endoscopic Microsurgery). As described above, the conventional TEM suffers from the following problems. For example, regarding the radial direction, the surgical field is restricted to only 180 degrees on the bottom surface of the rectum/colon due to the interference between the sheath and the forceps. Regarding the depth direction, the surgical field is restricted to a straight range that can be provided by the straight sheath SH, i.e. a straight range up to an S-shaped portion of the rectum. In this regard, as shown in FIG. 12A, the treatment system of the present embodiment eliminates or dramatically reduces these restrictions. Thus, the treatment system of the present embodiment can facilitate the approach to the lesion LS located at a curved portion, such as the sigmoid colon, of the rectum, while ensuring sufficiently large surgical field. Accordingly, high sufficiency is ensured in the operation of giving necessary treatment.

A system applicable to the conventional TEM is disclosed in JP-A-H07-313443 (published Dec. 5, 1995). This system is provided as an “insertion device used for inserting a lumen insert member”. The insertion device includes: a balloon arranged at the head portion of an endoscope; a jetting means for jetting a pressurized fluid backward (i.e. toward an operating portion side) from a portion on the head side; and a filling means for filling the area surrounding the jetting means with liquid. According to the insertion device, a closed space is created before the balloon, and the closed space is filled with liquid (e.g., physiological saline). In this state, a liquid jet stream is formed backward from the head of the endoscope. Thus, the jet stream formed in inserting the insertion portion of the endoscope into the rectum of a patient can apply a thrust to the insertion portion to further facilitate the insertion of the endoscope. Repeating the jet spraying in combination with the pushing operation of the insertion portion, the head portion of the endoscope is advanced past the sigmoid colon and can be positioned after the sigmoid colon. Thus, the sigmoid colon can be linearized to some extent.

However, the technique disclosed in JP-A-H07-313443 does not teach the establishment of the positional relationship between a lesion and a linearization range. Accordingly, although the disclosed technique may seem to resemble to the present embodiment, it is different in substance from the present embodiment.

(Modifications)

The first embodiment described above may be modified into various modes.

For example, the second balloon 4 as a closing member may be variously modified. Some modifications are set forth below.

(First Modification)

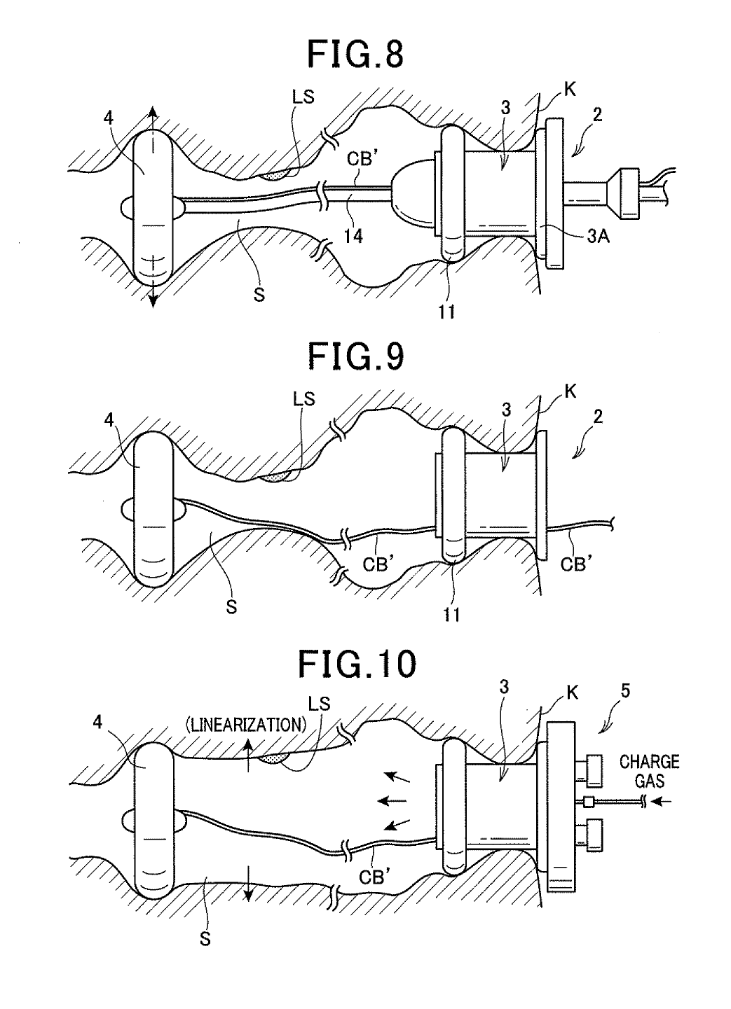

When the pressure of the gas charged into the treatment space S is increased, the second balloon 4, whose position has once been determined, is liable to move deeper into (move forward in the longitudinal direction of) the rectum because the inner wall of the rectum is soft. On the other hand, in order to firmly fix the second balloon 4 at a desired position, the inner pressure of the second balloon 4 may be increased. However, too much increase of the inner pressure tends to cause a negative effect, such as damaging the intestinal wall. To cope with this, two or more second balloons 4 may be used to disperse pressure. FIG. 13 shows an example of using two second balloons 24A and 24B.

FIGS. 14A and 14B show an example of using two second balloons 24A and 24B and of sucking the gas in the space between the balloons. As shown, the balloons 24A and 24B are connected to each other by a link 25 having holes HL. The link 25 communicates with a suction tube 21 connected to a suction machine, not shown. The gas in the space is sucked by the suction machine via the holes HL. Thus, when the gas is sucked in a state when the second balloons 24A and 24B are inflated, the intestinal wall is sucked inwards in the space between the balloons as shown in FIG. 14B. Thus, the suction of gas can more enhance the positioning function of the second balloons 24A and 24B.

Further, as shown in FIG. 15, three (or more) balloons 24A, 24B and 24C may be used.

(Second Modification)

In the first embodiment described above, a scope (rigid or flexible) having an image pickup function is used as a conveyer. Alternatively, however, a simple rigid pipe may be used as a conveyer.

### Second Embodiment

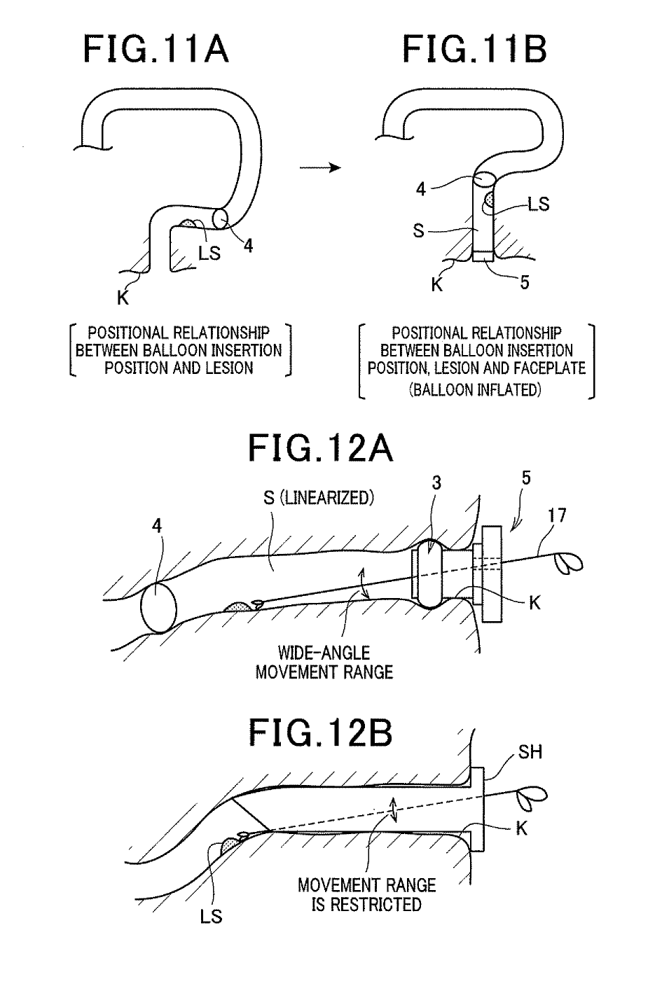

Referring to FIGS. 16 to 19, hereinafter is described a second embodiment of the present invention.

In the first embodiment and its modifications described above, the treatment space S is defined between the second balloon 4 (or a plurality of second balloons 4) positioned after the lesion LS and the first balloon 11 positioned before the lesion LS, and the defined treatment space S is linearized. In this case, as shown in FIG. 10, the gas charge/discharge tube CB′ connected to the second balloon 4 is left in the treatment space S. Therefore, the presence of the charge/discharge tube CB′ may disturb the treatment. To take measures against this, the second embodiment provides a method and system of treatment in which the charge/discharge tube CB′ does not have to be left in the treatment space S.

In the second embodiment, components identical with or similar to those in the first embodiment are given the same reference numerals for the sake of omitting or simplifying explanation.

FIG. 16 is a partially broken cross-sectional view illustrating a part of a treatment system for cutting away the method of treatment according to the second embodiment. As shown in FIG. 16, the treatment system includes a second balloon 104 which is provided with an engagement hole 104H, similar to the second balloon 4 in the first embodiment. The engagement hole 104H is provided with an O-ring 105. A guide pipe 106 mounted to the obturator 2 is pressed into the O-ring 105. Thus, the guide pipe 106 is detachably engaged with the second balloon 104 via the engagement hole 104H.

The guide pipe 106 may be an endoscope similar to the one described above, or may be a single rod-like member. When the guide pipe 106 is a single rod-like member, the rod-like member is formed of a transparent resin material and has flexibility. Although not shown, the rod-like member has a head in which a camera, such as a CCD camera, is incorporated. A cable used for transmitting the image signals of the camera is led from the operator's-side end portion of the rod-like member. The image signals are transmitted to a display, not shown, and displayed as images on the display.

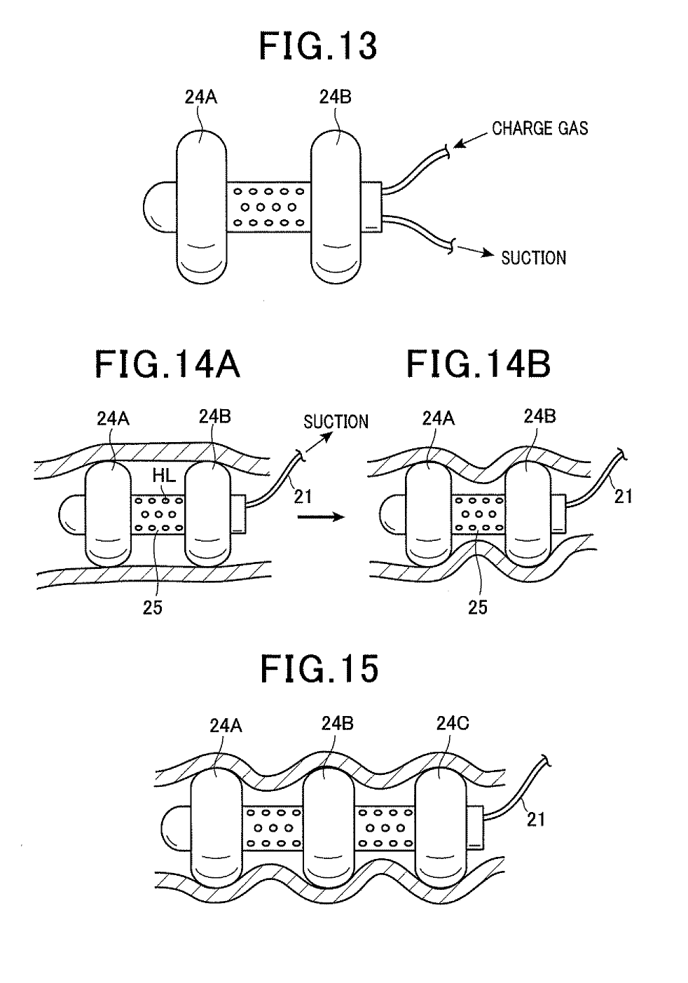

FIGS. 17 and 18 are diagrams illustrating a process of inflating/deflating the second balloon 104. As shown in FIG. 17, the second balloon 104 is provided with a gas charge/discharge hole 104J which is positioned near but radially outside the engagement hole 104H. The gas charge/discharge hole 104J is provided with a valve 107. Thus, as shown in FIG. 18, the gas charge/discharge hole 104J can be used for inserting the head of a local injection syringe 108.

The second balloon 104 has a balloon base portion 104A and an expansion/contraction portion 104B. The balloon base portion 104A has substantially an elliptical shape. The expansion/contraction portion 104B is integrally formed with the balloon base portion 104A and located radially outside the base portion 104A. The expansion/contraction portion 104B has a hermetically hollow space SP radially outside the base portion 104A, being isolated from the base portion 104A, and thus can be expanded/contracted in the radial direction. The gas charge/discharge hole 104J communicates with the hollow space SP.

The faceplate 5 used in the present invention is configured such that the guide pipe 106 and the local injection syringe 108 can be hermetically pushed into and pulled out of the rectum.

The remaining configuration is substantially identical with to or similar to that of the first embodiment.

Hereinafter is described a procedure of linearizing the treatment space S of the rectum, according to the present embodiment.

Similar to the above, in a state where the guide pipe 106 is engaged with the second balloon 104 via the engagement hole 104H, the second balloon 104 is inserted into the rectum (refer to steps S1 to S4 of FIG. 4. In a manner as described above, the operator can push the guide pipe 106 for insertion into the rectum, while observing the images on the display.

After completing the insertion, the operator inserts the local injection syringe 108 into the rectum via the through hole HL of the obturator 2 and pushes the head of the syringe 108 into the gas charge/discharge hole 104J via the valve 107. In this state, gas is charged from the local injection syringe 108 to expand the expansion/contraction portion 104B of the second balloon 104 (refer to step S5 of FIG. 4). Thus, the second balloon 104 hermetically closes the rectum at a desired position in the rectum. Then, the operator applies a little force to the guide pipe 106 to pull out the guide pipe 106 from the engagement hole 104H of the second balloon 104. At the same time, the operator pulls out the guide pipe 106 from the treatment space S in the rectum and also from the obturator 2. After that, processes and treatment similar to steps S6 to S10 of FIG. 4 are carried out.

When the treatment of the legion LS of the rectum is finished, the second balloon 104 is required to be deflated (refer to step S11 of FIG. 4).

In the present embodiment, the deflation is carried out as follows. First, the guide pipe 106 is again inserted into the treatment space S of the rectum via the faceplate 5. Further, the head portion of the guide pipe 106 is press-fitted to the engagement hole 104H of the second balloon 104. Thus, the guide pipe 106 is again able to catch the second balloon 104 in the rectum. Then, the local injection syringe 108 is inserted into the treatment space S of the rectum via the faceplate 5. Then, a needle portion at the head of the syringe 108 is inserted into the gas charge/discharge hole 104J of the second balloon 104 (see FIG. 18). In this state, the gas inside the hollow space SP is discharged by the local injection syringe 108 to deflate the second balloon 104. Thus, the positioning function of the second balloon 104 in the rectum is cancelled. At the same time, the second balloon 104 is brought into a state of being caught by the guide pipe 106. Accordingly, when the guide pipe 106 is pulled out, together with the sheath 3 and the faceplate 5, from the anus K, the second balloon 104 is also pulled out of the anus K.

Thus, the second embodiment is different from the first embodiment in that, in treating the lesion LS, the tube used for inflating or deflating the second balloon 104 is not left in the substantially hermetically created treatment space S of the rectum. In this way, the treatment space S is linearized and the space used for treatment is practically enlarged to thereby facilitate the treatment.

Some modifications applicable to the second embodiment are described.

(Third Modification)

The plurality of second balloons in a modification of the first embodiment described referring to FIGS. 13 to 15 may also be applied to the second embodiment. To this end, the hollow spaces SP of the two second balloons 24A and 24B, for example, shown in FIG. 13 may be permitted to hermetically communicate with each other via a channel, with one of the balloons being provided with the gas charge/discharge hole explained referring to FIGS. 17 and 18. Thus, by performing a gas-charging operation only once, the plurality of second balloons can be inflated or deflated. The same applies to the case where the number of the second balloons is three (see FIG. 15). Thus, this modification can enjoy both of advantages of: not leaving the gas charge/discharge tube in the treatment space S as in the second embodiment; and firmly establishing fixation of the plurality of second balloons at respective positions.

(Fourth Modification)

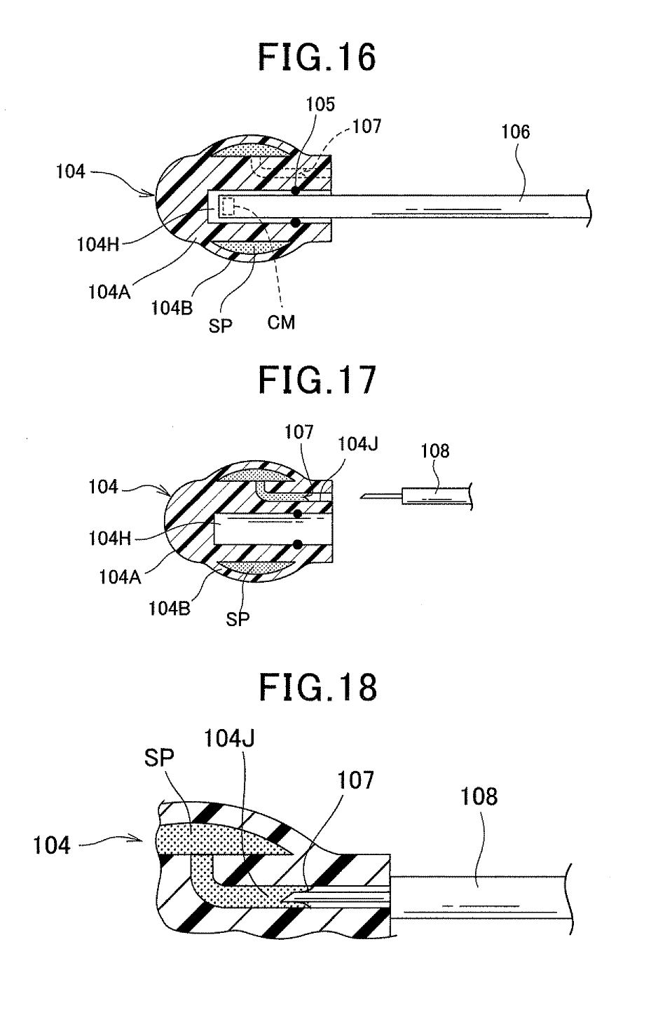

FIG. 19 shows a fourth modification of the second embodiment. The fourth modification is obtained by further modifying the third modification. Specifically, the method of placing the second balloon 104 in the rectum according to the third modification is further modified in the fourth modification. Therefore, in the fourth modification, the components identical with or similar to those in the third modification are given the same reference numerals for the sake of simplifying explanation.

Similar to the above, the second balloon 104 shown in FIG. 19 has an engagement hole 104H. The head portion of a columnar guide pipe 116 made of a resin material and having flexibility is press-fitted to the engagement hole 104H. The guide pipe 116 has a hollow space 116H into which an insertion portion 117 of an endoscope (not shown) is loosely inserted. Accordingly, in inserting the second balloon 104 into the rectum, the guide pipe 116 engaged with the second balloon 104 by the press-fitting is pushed into the rectum together with the insertion portion 117 of the endoscope. Since a camera CM is mounted to the head of the insertion portion 117 of the endoscope, the images captured by the camera CM can be referred to for the insertion of the second balloon 104.

On the other hand, when the second balloon 104 is solely placed in the rectum, the sole second balloon 104 is inflated as described above. Then, the action of pushing the second balloon 104 by pushing the insertion portion 117 of the endoscope is conducted in parallel with the action of pulling out the guide pipe 116 against the press-fitting resistance. As a result, the second balloon 104 stays at its position by the pushing force, while the guide pipe 116 is pulled out of the engagement hole 104H of the second balloon 104. After that, both of the insertion portion 117 of the endoscope and the guide pipe 116 are pulled out of the rectum via the faceplate 5. Thus, finally, the second balloon 104 alone is placed at a desired position in the rectum, similar to the second embodiment and the third modification.

(Fifth Modification)

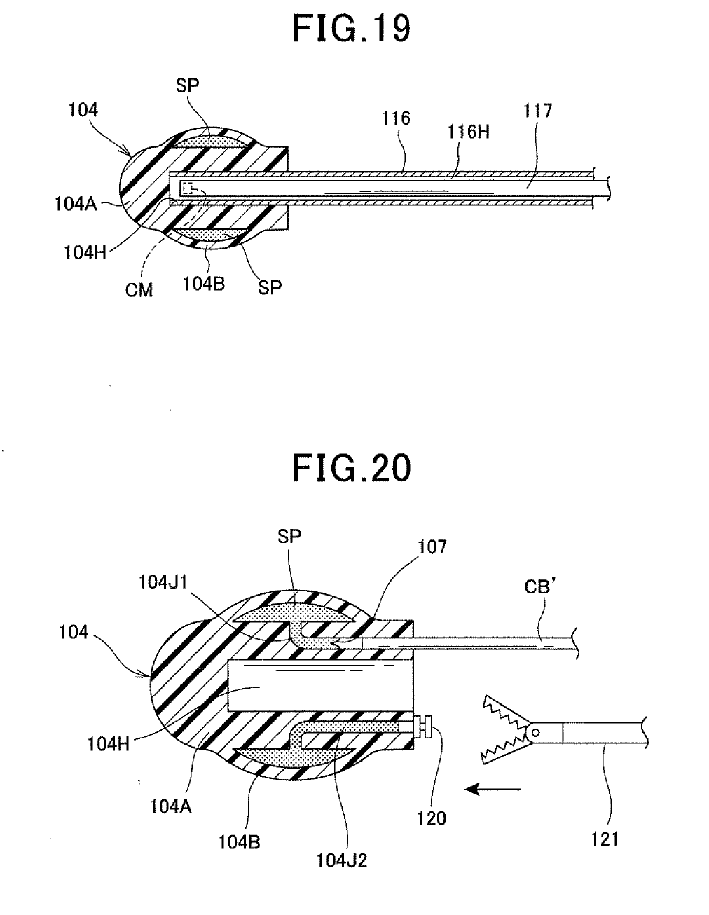

FIG. 20 shows a fifth modification of the second embodiment. In a treatment system of the fifth modification, the tool used for charging gas into the hollow space SP of the second balloon 104 is different from the tool used for discharging gas from the hollow space SP.

Specifically, as shown in FIG. 20, the treatment system of the fifth modification includes a gas charge hole 104J1 and a gas discharge hole 104J2 used for charging and discharging gas, respectively. The charge hole 104J1 is provided with a check valve 107 therein, while the discharge hole 104J2 is provided with a stopper 120. A tube CB′ similar to the above is inserted into the charge hole 104J1 to charge gas into the hollow space SP via the check valve 107. After completion of charging gas, the tube CB′ is pulled out. In this case, since the check valve 107 provided in the charge hole 104J1 prevents the gas from leaking out of the hollow space SP, the second balloon 104 is inflated and increases its diameter.

On the other hand, in discharging gas, the stopper 120 of the discharge hole 104J2 is removed using forceps 121. Accordingly, gas is discharged from the hollow space SP of the second balloon 104 that has been inflated. As a result, the second balloon 104 is deflated, with its positioning function being cancelled.

The structure used for charging/discharging gas to/from the second balloon, as illustrated in FIG. 20 may be combined with the structure used for placing the second balloon in the rectum, as illustrated in FIG. 19. Further, of the structures shown in FIGS. 16 to 18, used for charging/discharging gas, the structures used for discharging gas may each be replaced by the gas-discharging structure shown in FIG. 20. In this way, in the fifth modification as well, advantages similar to those of the embodiments and modifications described above are obtained.

(Sixth Modification)

The shape of the second balloons 4 and 104 related to the first and second embodiments and their modifications is not limited to the one described above, but may have a long elliptical shape. The long elliptical shape can provide a plane contact, instead of a point contact, between the second balloon and the intestine wall. Owing to the plane contact, the second balloon hardly moves along the intestine wall and enhances is positioning function. Alternatively, the shape of the second balloons 4 and 104 may have the shape of a gourd. A gourd-shaped second balloon comes into engagement with the fold of the intestine wall and thus is able to prevent itself from inclining to thereby firmly establish its position with respect to the intestine wall. Thus, the positioning function of the second balloon is enhanced.

The present invention is not limited to the configurations of the embodiments and their modifications described above. The present invention may be implemented in combination with well-known configurations of conventional art for the provision of more appropriate modes, within a range not departing from the spirit of the present invention.

What is claimed is:

1. A method of treating a lesion in a tubular organ that communicates with a natural orifice of a subject, wherein the method comprises:
a first step of closing or substantially closing a channel of the tubular organ at a desired first position on a far side of the lesion;
a second step of closing or substantially closing a channel of the tubular organ at a second position on a near side of the lesion, the second position being near the natural orifice;
a third step of linearizing the tubular organ by sending a fluid into a closed space of the tubular organ, the closed space being created between the first and second positions by bringing the tubular organ into a closed or substantially closed state through the first and second steps; and
a fourth step of treating the lesion by inserting treatment tools into the linearized closed space.

2. The method according to claim 1, wherein:
the first step includes a step of advancing a first inflatable sealing member in a deflated state to the first position via the natural orifice and a step of inflating and placing the first sealing member at the second position after the advancement;
the second step includes a step of placing a second sealing member at the second position, the second sealing member having treatment-tool guide lumens into which treatment tools can be inserted;
the third step includes a step of sending gas as the fluid; and
the fourth step is a step of introducing the treatment tools into the closed space via the treatment-tool guide lumens of the second sealing member to treat the lesion located in the closed space.

3. The method according to claim 2, wherein the natural orifice is the anus of the subject and the tubular organ is the sigmoid colon.
